(19) United States
(12) Patent Application Publication
(10) Pub. No.: US 2007/0261268 A1
(43) Pub. Date: Nov. 15, 2007

(54) Insole to reduce plantar pressure

(76) Inventor: Hienvu Chuc Nguyen, Fair Oaks, CA (US)

Correspondence Address: HIENVU CHUC NGUYEN, 3926 Oak Hurst Circle, Fair Oaks, CA, 95628, US

(21) Appl. No.: 11/430,210

(22) Filed: May 9, 2006

(51) Int. Cl. A43B 7/06; A43B 13/38; A61F 5/14

(52) U.S. Cl. 36/44; 36/140; 36/3 B

(57) ABSTRACT

A new insole is consisting of shock absorbing and shear reducing composite layers of Poron or soft EVA for the bottom layer, Plastazote or cushioned polymer gel for the middle layer, and a closed cell Neoprene top cover. Through out the insole, there are evenly spaced 3/16″ holes through only the bottom and the middle layer of the insole. These holes are ½″ away from each other and in square patterns. The holes will significantly reduce the direct plantar pressure and shear stress dynamically exerting on the plantar skin upon loading. When there is a focal point of pressure, the holes in the bottom 2 layers will be distorted or stretched to the direction of the pressure which will also allow the insole material to distorted or “give” resulting in reduction of the peak plantar pressure.

### CROSS-REFERENCE TO RELATED APPLICATIONS

Not applicable

### BACKGROUND OF THE INVENTION

There are 16 million people or 5.9% of the population in the United States that have diabetes. Many people first become aware that they have diabetes when they develop one of its major complications such as blindness, heart disease, stroke, peripheral vascular disease, and numbness or neuropathy in the feet. High blood sugar level also affects the body immune system and cause delayed wound healing.

Diabetic foot complications are the most common cause of non-traumatic lower extremity amputations in the United States. The risk of lower extremity amputation is 15 to 46 times higher in diabetics than in normal persons, and the majority of diabetic foot complications begin with the formation of skin ulcers on the bottom of the foot.

One of the main causes for diabetic ulceration is the increase in plantar pressure on the bottom of the foot; especially, the forefoot and the heel area. Foot deformities, which are common in diabetic patients, lead to focal area of high pressure. When an abnormal focus of pressure is coupled with lack of sensation, a foot ulcer can develop. Therefore, off-loading plantar foot pressure is an important component in treating diabetic foot ulcerations.

### DESCRIPTION OF PRIOR ARTS

There are many off-loading techniques and devices available, each having specific applications according to the anatomic location of the wound. Off-loading devices made for non-ambulatory use include airflow mattresses, soft padding for the bed and wheelchair, and heel protectors such as the multipodus boot that suspends the limb to completely remove pressure from the problem area.

For ambulatory patients with plantar foot ulcerations, the ultimate off-loading device is a total contact cast, which acts to transfer weight away from the foot and redistributes the forces of weight bearing proximally onto the leg. A removable cast walker device performs much of the same off-loading as a total contact cast but is often more easily tolerated by the patient because it is removable for bathing and for daily wound care. Other off-loading but less ideal devices include a half-shoe (or so-called “wedge shoe”), a postoperative/surgical shoe with soft accommodative padding layer. A randomized clinical study was conducted to compare the effectiveness of total-contact casts, removable cast walkers, and half-shoes to heal neuropathic foot ulcerations in individuals with diabetes. The study reports that a significantly higher proportion of patients were healed in 12 weeks in the total contact cast group when compared with the two other modalities (89.5% vs. 65%, and 58.3%) (Off-loading the diabetic foot wound: a randomized clinical trial, Diabetes Care 2001 November; 24(11):2016)

U.S. Pat. No. 5,197,942 (Brady) describes wearable foot orthoses with wound aperture to offload plantar pressure when a patient walk. However, most diabetic patients with foot ulceration have insensate foot and orthoses with hard material could create additional plantar foot ulceration.

U.S. Pat. No. 5,329,705 (Grim) describes removing grids of removable resilient hexagon elements in the insole to offload the plantar foot pressure. However, this practice will subsequently transferring peak plantar pressure to other part of the foot. Furthermore, as the pressure is reduced through the aperture (from removing the hexagon elements) it will put additional pressure around the wound edge and subsequently creating more skin undermining and calluses around the wound.

U.S. Pat. No. 5,483,757 (Frykberg) describes insole with preferably multiple evenly spaced holes extends through for breathing. These are limited number of small holes with the main purpose for air circulation through the insole. These hole design has no effective peak plantar pressure reduction.

U.S. Pat. Nos. 5,797,862, and 6,083,185 (Lamont) describes diabetic boot and insole consisting of an upper layer plastazote and lower layer of poron material which laminated together with additional metatarsal and scaphoid paddings to offload foot pressure. This kind of material is commercially available in sheets which can be cut out to fit into a shoe or boot. However, it still does not have the offloading capabilities such as the new invention (see testing below).

U.S. Pat. No. 5,799,413 (Argyris) describes small round air ventilation channels through the top cover which is made of thin leather which has no cushioning effects because the material is too thin in thickness, does not give readily when there is focal point of pressure.

U.S. Pat. No. 5,921,003 (Kim) describes insole with round holes through the top layer with the main purpose for distributing hygienic chemical from a cartridge in the heel. The holes are for ventilation purpose only, too thin in thickness, and has no elastic properties to help reduce a focal point of pressure.

U.S. Pat. No. 5,845,418 (Chi) describes insole with ventilation holes on the top layer which opens to air chambers in the bottom layer. Again, the holes are for ventilation only, too thin in thickness, and has no elastic properties help reduce a focal point of pressure.

### SUMMARY OF THE INVENTION

The main purpose of this invention is to develop a new insole design which can significantly reduce the plantar pressure under the forefoot and heel, and can be used as replacement insole for the diabetic shoe, diabetic healing shoe, and removable cast walker.

This new insole is consisting of shock absorbing and shear reducing composite layers of Poron or soft EVA for the bottom layer, Plastazote or cushioned polymer gel for the middle layer, and a closed cell Neoprene top cover. These materials are commercially available in sheets.

The main advantage and uniqueness of this invention, comparing to other existing insoles, are the evenly spaced 3/16″ holes through only the bottom and the middle layer of the insole. These holes are ½″ away from each other and in square patterns throughout the insole. There is a closed cell Neoprene top cover. The holes will significantly reduce the direct plantar pressure and shear stress dynamically exerting on the plantar skin upon loading. When there is a focal point of pressure, the holes in the bottom 2 layers will be distorted or stretched to the direction of the pressure which will also allow the insole material to distorted or “give” resulting in reduction of the peak plantar pressure and the associated shear stress. See FIG. 3. This will also eliminate any pressure transferring problems as encountered in other insoles. Removing the pressure will allow the insole material to return back to the original state.

Therefore, dynamic direct plantar pressure and shear stress can be significantly reduced by these holes.

This insole can also be used to offload plantar pressure of deformed foot which has bony prominences due plantar fat pads atrophy in people with rheumatoid arthritis.

This new insole, when used in conjunction with a shoe or with removable cast walker, will replace the total contact cast, which is the ultimate off-loading device for treating diabetic foot ulcerations.

### BRIEF DESCRIPTION OF THE SEVERAL VIEWS OF THE DRAWING

FIG. 1 is top view of the insole.

FIG. 2 is the right side view of the insole.

FIG. 3 is side view shows how the material gives toward the focal pressure point P.

### DETAILED DESCRIPTION OF THE INVENTION

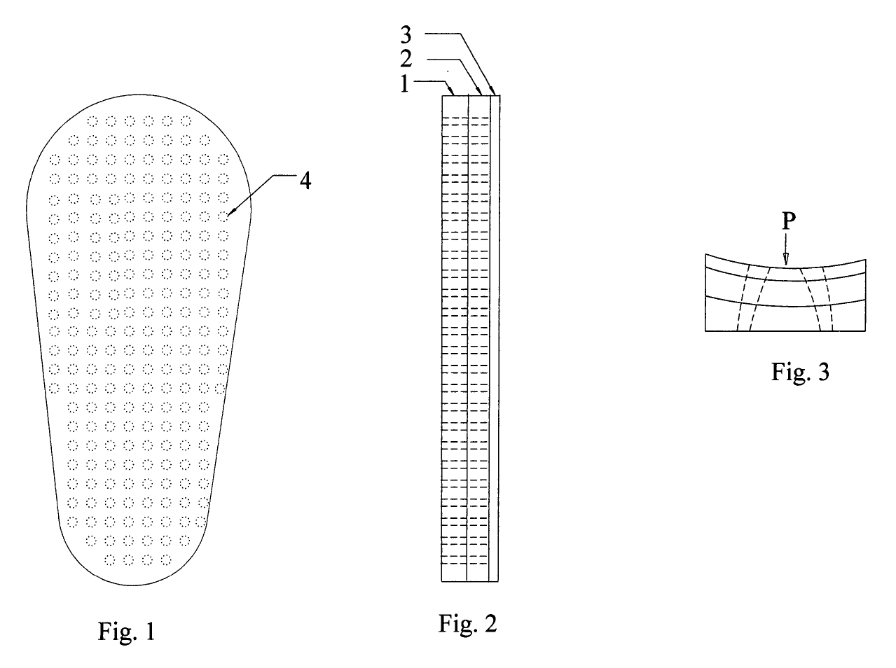

This invention of the off-loading insole for diabetic ulceration consists of a shock absorbing composite layers insole, which also has additional capability to reduce the peak plantar foot pressure and shear stress on the plantar skin by evenly spaced 3/16″ diameter holes through only the bottom 2 layers of the insole. These holes are ½″ away from each other in square patterns. See FIG. 1, and FIG. 2.

The insole main shape can be of a foot, existing shoe insole, or the outline of a post surgical shoe or removable cast walker.

The bottom layer 1 material is soft EVA with a thickness of ¼″ which has excellent shock absorption, high direct impact performance, and high resiliency.

The middle layer 2 material can be either pink Plastazote with a thickness of ¼″ to mainly absorb the direct plantar pressure and to reduce the shearing stress.

The top layer 3 material is closed cell Neoprene with a thickness of ⅛″ and is used as the top cover for the insole.

All three materials are glued together to form a composite layered insole. These materials are commercially available.

There are 3/16″ diameter round holes 4 through the bottom 2 layers of the insole. There holes are ½″ away from each other in a square pattern.

Main shear stress reduction is accomplished by the bottom 2 layers since they have the elastic property to “slide” or “give” when the direction of force is parallel or near parallel to the insole surface.

When there is a focal point of pressure, the holes will be distorted or stretched to the direction of the pressure which will also allow the insole material to distorted or “give” resulting in reduction of the peak plantar pressure and the associated shear stress. Removing the pressure will allow the insole material to return back to the original state. Therefore, additional dynamic direct plantar pressure and shear stress can be significantly reduced by these holes. See FIG. 3.

Plantar pressure studies have been completed and it was determined that the holes must be 3/16″ in diameter and must be spaced ½″ away from each other in square patterns, and only though the bottom 2 layers of a composite insole made of EVA and Plastazote layers with at least ½″ total thickness. Any other smaller hole size, or smaller spacing, or thinner material will not achieve the peak plantar pressure reduction. This is the main different than all other existing insole designs with though-and-through holes for ventilation purpose.

Results of the Peak Plantar Pressure of Various Insoles

We conducted a study to determine the plantar pressure under the medial forefoot and under the heel using:
1. Plain surgical shoe
2. Laminated insole with ¼″ EVA and ¼″ pink Plastazote. This is the normal insole material for diabetic shoe.
3. Laminated insole with ¼″ soft EVA for the bottom layer, ¼″ pink Plastazote for the middle layer, and ⅛″ Spenco for the top layer. One has no hole and another one has 3/16″ holes drilled through all layers of the insole and are evenly spaced at ½″.

The plantar pressures were measured using the F-Scan in-shoe pressure measurement system (TekScan, South Boston, Mass.). The test was performed on a treadmill at a walking speed of 1.5 mph with a female subject weights 135 lbs and wearing a surgical shoe with and without the insoles above. Data were collected and tabulated as follow:

| | Plain Surgical Shoe |
|---|---|
| | Pressure |
| Pressure Under Medial Forefoot | 15 psi (10.35N/Cm2) |
| Pressure Under Heel | 20 psi (13.8N/Cm2) |

Insole with EVA and pink Plastazote and surgical shoe

| | Number 1 Insole |
|---|---|
| | Without Holes |
| Pressure Under Medial Forefoot | 14 psi (9.66N/Cm2) |
| Pressure Under Heel | 18 psi (12.42N/Cm2) |

Insole with ¼″ soft EVA for the bottom layer, ¼″ pink Plastazote for the middle layer, and ⅛″ Spenco for the top layer, and surgical shoe

| | Number 2 insole | |
|---|---|---|
| | Without Holes | With Holes |
| Pressure Under Medial | 13 psi (8.98N/Cm2) | 10 psi (6.9N/Cm2) |
| Forefoot | | |
| Pressure Under Heel | 17 psi (11.73N/Cm2) | 15 psi (10.35N/Cm2) |

As indicated in the tables above, the peak plantar pressure was further reduced by using insoles with evenly spaced holes.

Similar study conducted by Layery LA and cohorts in “Reducing Dynamic Foot Pressures in High-risk Diabetic Subjects With Foot Ulcerations” (Diabetes Care 19(8): 818-821, 1996) reports mean peak pressure for ulcers under the 1^{st} metatarsal heads (medial forefoot) for the Total Contact Cast, DH Pressure Relief Walker (Royce Medical, Camarillo, Calif.), Aircast Pneumatic Diabetic Walker (Aircast, Summit, N.J.), Extra Depth Shoe as 7 N/Cm2, 8 N/Cm2, 12.3 N/Cm2, and 39.5 N/Cm2 respectively.

Another study conducted by Armstrong DG and cohorts in “Total Contact Casts and Removable Cast Walkers” (J Am Podiatric Medical Association 89(1): 50-53, 1999) reports peak plantar heel pressure for the Total Contact Cast, DH Pressure Relief Walker Aircast Pneumatic Diabetic Walker, and PW Minor Extra Depth Shoe (PW Minor and Son, Batavia, N.Y.) as 18 N/Cm2, 19 N/Cm2, 20 N/Cm2, and 25 N/Cm2 respectively.

Comparing to the above studies for peak plantar pressure under the medial forefoot and the plantar heel, there is not a substantial difference between the new invention insole and the Total Contact Cast, which is a gold standard for offloading diabetic foot ulceration, to offload the medial forefoot (6.9 N/Cm2 vs 7 N/Cm2). However, the new insole is much better to offload the plantar heel pressure (10.35 N/Cm2 vs 18 N/Cm2).

What I claim is:

1. Evenly spaced 3/16″ diameter round holes punched through only the bottom and the middle composite layers of EVA of ¼″ in thickness, and Plastazote in ¼″ in thickness, throughout the insole to reduce peak plantar foot pressure and shear stress. There is a ⅛″ closed cell Neoprene top layer.

2. These holes are ½″ away from each others and in square patterns throughout the insole.

3. When there is a focal point of pressure, the holes in the bottom 2 layers will be distorted or stretched to the direction of the pressure which resulting in reduction of the peak plantar pressure and the associated shear stress. This will also eliminate any pressure transferring problems as encountered in other insoles. Removing the pressure will allow the insole material to return back to the original state.
